(19) United States
(12) Patent Application Publication
(10) Pub. No.: US 2012/0272964 A1
(43) Pub. Date: Nov. 1, 2012

(54) MOBILE RESPIRATOR

(75) Inventors: Judith LÖSER, Lubeck (DE); Götz KULLIK, Lubeck (DE); Ulf ZSCHERNACK, Ahrensburg (DE); Tobias OTTE, Lubeck (DE)

(73) Assignee: DRÄGER MEDICAL GMBH, Lubeck (DE)

(21) Appl. No.: 13/350,110

(22) Filed: Jan. 13, 2012

(30) Foreign Application Priority Data
Apr. 27, 2011 (DE) 10 2011 018 671.9

(51) Int. Cl. A61M 16/00; A61M 16/08

(52) U.S. Cl. 128/204.23

(57) ABSTRACT

A respiration device with a respirator (3) and a sensor unit (8) close to the patient has a display unit (7) for measured parameters, with a breathing gas tube (5) between the respirator (3) and the sensor unit (8). A bidirectional data connection is provided between the sensor unit (8) and the respirator (3). The physiological measured values important for the respiration are recognized in the vicinity of the patient in a need-adapted manner. A configuration device is provided, by which presettable display fields can be set for the measured parameters on the display unit (7).

### CROSS REFERENCE TO RELATED APPLICATIONS

This application claims the benefit of priority under 35 U.S.C. §119 of German Patent Application DE 10 2011 018 671.9 filed Apr. 27, 2011, the entire contents of which are incorporated herein by reference.

### FIELD OF THE INVENTION

The present invention pertains to a mobile respirator for emergency respiration and respiration during transportation with display of measured values in the vicinity of the patient.

### BACKGROUND OF THE INVENTION

A respiration device of the type mentioned is known from DE 10 2008 028 662 A1. The prior-art device comprises a respirator, which is connected via a breathing tube to a patient interface, which comprises a breathing bag. A sensor means for measuring respiration parameters with a display unit is located at the patient interface in order to communicate current values to the user. The display unit comprises, furthermore, visualizing elements, which are used to show the degree of filling of the lungs or changes in a parameter. The inspiratory and expiratory volume, respiration pressure and respiration rate are measured as respiration parameters in order to determine variables derived therefrom, such as the respiratory minute volume.

A respiration device, in which a sensor means is connected via a breathing tube to a respirator and a signal line, which is used for energy and data exchange between the sensor means and the respirator, extends along the breathing tube, is known from DE 103 12 881 B3. The sensor means is designed to measure temperature, humidity, gas flow, breathing gas concentration and breathing gas pressure.

It is difficult to clearly display the parameters relevant for the current respiration to the user because of the large number of measured variables and variables derived from measured variables. In addition, mechanical respiration supported by the respirator may also occur besides a pure manual respiration, which the user carries out by means of the manual breathing bag in the immediate vicinity of the patient. In case of manual respiration, only the fresh gas necessary for the respiration is delivered by the respirator, while the respirator additionally assumes the control of the inspiration phases and expiration phases in case of mechanical respiration. This leads to different requirements on the measured variables, which must be communicated to the user. The user focuses on the patient's face, especially in the special situation of emergency respiration, in order to be able to observe in the critical situation the color of the skin and of the lips, reflexes of the pupils and the measured parameters displayed on the display unit in the vicinity of the patient on the display unit of the sensor unit. Measured parameters, which are displayed or outputted at the respirator itself, are not within the field of view of the user.

### SUMMARY OF THE INVENTION

A basic object of the present invention is to improve a respiration device of the type mentioned such that the physiological measured values, which are important for the respiration, can be recognized in the vicinity of the patient in a need-adapted manner.

According to the invention, a respiration device is provided comprising a respirator and a sensor unit to be positioned close to a patient, the sensor unit comprises a display unit for measured parameters. A breathing gas tube is provided between the respirator and the sensor unit. A bidirectional data connection is provided between the sensor unit and the respirator. A configuration means for providing presettable display fields for displaying measured parameters on the display unit is provided.

Provisions are made according to the present invention for providing at a respiration device a sensor unit located close to the patient, which has a display unit for outputting physiological measured values such as inspiratory and expiratory gas flows, airway pressure, carbon dioxide concentration at the site of measurement itself, i.e., in the vicinity of the patient at the breathing gas port of the breathing mask or of the endotracheal tube. This measured data display also functions without the activation of the mechanical respiration at the respirator in a special measuring mode of the respirator, so that no alarms are generated as a consequence of the absence of mechanical respiration.

The sensor unit and the display unit are connected to the respirator via a bidirectional data connection for energy supply and for data exchange with the respirator. In a preferred embodiment of the present invention, all sensors at the breathing gas port are arranged in a sensor unit, so that only the electric power is supplied from the respirator and the sensor unit transmits the measured values to the respirator and displays these on a display unit of its own.

The electric connection along the breathing gas tube can be mechanically integrated in the tube, and electric plug-and-socket connections to the sensor unit and to the respirator are present. As an alternative to an electric plug-and-socket connection, an inductive coupling may be provided as well.

According to the present invention, the respiration device has a configuration means, by which presettable display fields can be displayed on the display unit. These are preformatted display fields, which are stored in a memory and are activated on the display unit depending on the form of respiration selected, such as manual respiration, with the manual breathing bag or mechanical respiration with the respirator. As it is difficult for the user such as a paramedic or emergency physician in the stressful situation of an emergency to perform the respiration adapted to the patient, the configuration means offers support in the display of the measured values in such a way that only the measured values that are relevant in the current form of respiration are outputted on the display unit. The display fields may be preset in an automated manner as a function of the current form of respiration, or the user is offered a selection of display fields, from which he can select the desired display field. The user has the possibility to vary the manual respiration, depending on the individual situation of the particular patient, until the measured values confirm a selected therapy and the patient is stabilized, so that he can then continue to be respirated mechanically. The user now can continue to use the measured values of the manual respiration for setting the machine parameters in a partly automated manner, and he does not have to re-enter them. Depending on the selected form of respiration, the measured values determined with the sensor unit located close to the patient can be used to control the respiration, for example, the flow trigger to recognize phases of respiration during spontaneous breathing. Important measured values are the respiration rate, peak pressure PIP and end-expiratory carbon dioxide concentration etCO₂, as well as expiratory tidal volume VTe and expiratory minute volume MVe.

The oxygen is supplied during manual respiration via an oxygen cylinder connected to the respirator. As an alternative, it is possible to supply the oxygen of the manual breathing bag via the respirator in such a way that oxygen is supplied at a pressure set at a fixed value, e.g., 5 mbar in the CPAP respiration mode, and the manual breathing bag is thus always filled regardless of the quantity removed. The pressure regulation of the respirator is thus advantageously used. In addition, the oxygen consumption can be determined by means of the flow measurement of the respirator in order to perform a precalculation of the remaining service life of the device. A corresponding display field is provided on the display unit for this application as well.

To adapt the respiration, manual and mechanical, it may be useful to enter in the device information on the patient, e.g., body weight, age and the state of the lungs as visible from the outside, which the user can recognize. The respirator determines useful respiration parameters from this, compares these with the respective instantaneous measured values and informs the user of deviations or suggests alternative parameter settings for the mechanical respiration to the user by means of a selected display field on the display unit.

The configuration means for the display fields is advantageously either part of the sensor unit or part of the respirator. The configuration means either selects the corresponding display field from a library of display fields on the basis of the current form of respiration and sets hereby the measured values to be displayed on the display unit. As an alternative, it is possible to offer the user a selection of display fields for selection. The configuration means may be a program module in the microprocessor of the sensor unit or of the respirator, which analyzes the current form of respiration and selects a corresponding display field or offers suitable display fields for selection.

The display unit is advantageously designed as a freely programmable display field, so that a plurality of different display fields can be embodied. The display field may be preferably designed on the basis of the LC or LED technology.

The display fields are advantageously structured such that a first group of display fields is provided for the manual respiration and a second group for the mechanical respiration.

The sensor unit can be advantageously carried along for transportation on a parking holder of the respirator. Such a transport unit comprising a respirator and a sensor unit can also be used to switch the respirator on and off via the sensor unit. The respirator is switched on as soon as the sensor unit is removed from the parking holder and is switched off as soon as the sensor unit is plugged onto the parking holder. The course of the respiration therapy at the accident site is thus supported in an especially simple manner and unnecessary actions are avoided.

The display unit at the sensor unit may have various designs. The measured values may be displayed in numeric form, e.g., respiration rate, or in curves, for example, the curve describing the carbon dioxide concentration, or in the form of intuitively comparable representations similarly to an artificial horizon in the cockpit of an aircraft, or in a very simple form in the form of a green-yellow-red traffic light. The representations in the form of a traffic light or of an artificial horizon permit a simple comparison with target values, which arise, e.g., from the patient's age and the patient's body weight. It is also possible in this manner to represent the patient's status and the status of the mechanical respiration on the display of the sensor unit during mechanical respiration.

It is advantageous, in general, to make the representation of the respiration parameters for manual respiration and mechanical respiration very similar or identical, so that simple operation and monitoring is made possible especially in the stress situation at the accident site.

Besides, it may be useful to also display parameters of other sensors, e.g., oxygen saturation from a finger clip, on the display at the patient's face in order to thus obtain a more comprehensive picture of the patient's state of breathing. These measured values may originate from other measuring devices, e.g., an ECG, blood pressure and oxygen saturation monitor, and be transmitted, e.g., in a wireless manner to the sensor unit.

In addition, an alarm display as well as an operational control for silencing possibly occurring acoustic alarms on the display at the patient's face are useful.

The operation may be advantageously carried out, in general, by means of operational controls in the display unit by means of the so-called “touch screen” technology.

To support cardiac massage during resuscitation, the respirator may be used such that the sensor unit measures and displays the frequency of pressure applied to the heart as gas pressure and gas flow variations in the lungs and thus informs the user of the effective frequency. In addition, it is currently taught that the administration of two respiration strokes after 30 cardiac compressions is a useful procedure in resuscitation. The sensor unit can measure and display the number of cardiac compressions and send a prompt for respiration after 30 compressions. The two breaths may be triggered at the sensor unit, e.g., by means of a button, so that the user can hold and seal the breathing mask and trigger the breaths via the button at the sensor unit in the immediate vicinity of the breathing mask at the same time.

An exemplary embodiment is shown in the drawings and will be explained in more detail below. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a schematic view showing a respiration device with a respirator and a sensor unit;

FIG. 2 is a schematic view showing the respiration device in accordance with FIG. 1 with manual breathing bag and gas supply via the respirator;

FIG. 3 is a schematic view showing the respiration device according to FIG. 2 with direct gas supply from an oxygen cylinder;

FIG. 4 is a schematic view showing the sensor unit with the display unit; and

FIG. 5 is a schematic view showing details of the sensor unit.

### DESCRIPTION OF THE PREFERRED EMBODIMENTS

Referring to the drawings in particular, FIG. 1 shows a patient 1 and a user 2 in the form of a paramedic or emergency physician, who is located at the patient, and a respirator 3 with an oxygen cylinder 4. The oxygen cylinder 4 supplies the respirator 3 with oxygen, which reaches the patient 1 through the breathing tube 5, optionally mixed with ambient air. The breathing gas is fed by means of a patient connection such as an endotracheal tube, not shown specifically, into the trachea of the patient 1. A sensor unit 8 with a display unit 7 in the vicinity of the patient 1 is used to monitor the respiration.

Sensor unit 8 measures the airway pressure, breathing gas flow and carbon dioxide concentration. The display values important for the respiration, such as the maximum inspiratory pressure (Peak Inspiratory Pressure, PIP), the volume of the expired breath (expiratory Tidal Volume, VTe), the respiratory rate (RR), and the carbon monoxide concentration at the end of expiration (end-expiratory Carbon Dioxide Concentration, etCO₂) are determined from these values and displayed.

The sensor unit 8 is supplied with power via a cable 6, which also transmits the measured values into the respirator 3.

Cable 6 and the breathing gas tube 5 are connected to one another by means of a small number of easy-to-detach holding elements 9. The sensor unit 8 and the respirator 3 together form a respiration device 100.

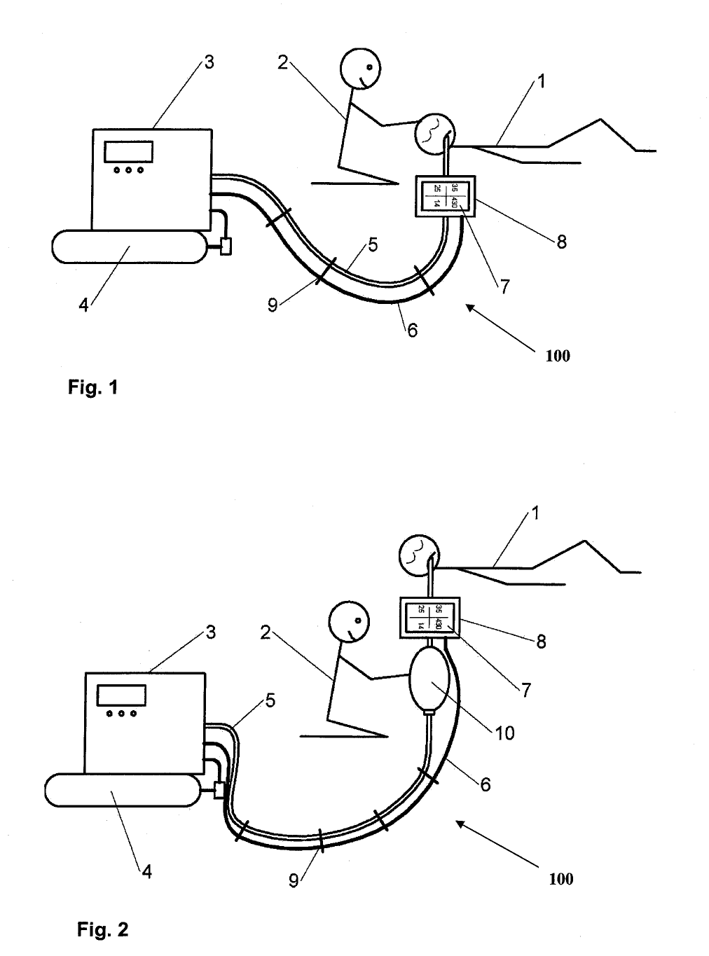

FIG. 2 shows a use of the respirator 3 during manual respiration by means of a manual breathing bag 10. This form of therapy is often performed prior to mechanical respiration. The manual breathing bag 10 is connected to a connector, not shown more specifically, in the area of the sensor unit 8 and receives oxygen from the respirator 3 by means of the breathing gas tube 5. Cable 6 for supplying the sensor unit 8 is connected to the breathing gas tube 5 by means of a small number of easy-to-detach holding elements 9. In this application, the respirator 3 delivers breathing gas under a fixed pressure to fill the breathing bag 10. For example, the so-called CPAP breathing mode is set for this at the respirator 3.

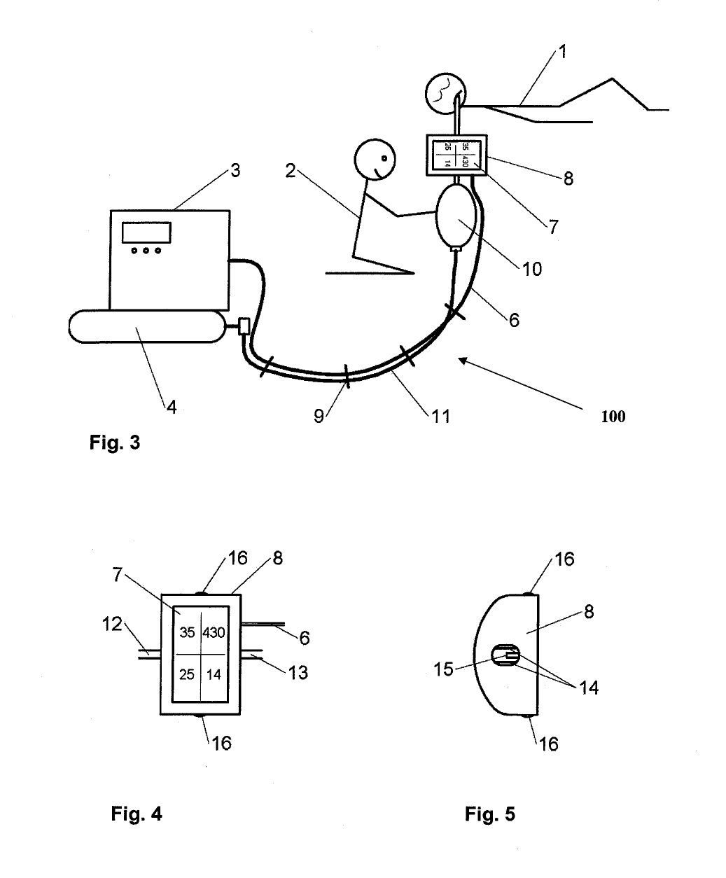

FIG. 3 illustrates another use of the respirator 3 during manual respiration by means of a breathing bag 10. The breathing bag 10 is connected to the sensor unit 8 and receives oxygen from the oxygen cylinder 4 of the respirator 3 by means of the O2 supply tube 11. The cable 6 for supplying the sensor unit 8 is separated from the breathing gas tube 5 in this case and extends in parallel to the O2 supply tube 11. Cable 6 and O2 supply tube 11 are connected to one another by means of a small number of easy-to-detach holding elements 9.

FIGS. 4 and 5 show details of the sensor unit 8. A tube, not shown more specifically, which has a patient port 12 and a manual breathing gas port 13 as port elements, extends through the sensor unit 8. The inspired gas and expired gas flow through the tube. The endotracheal tube, not shown more specifically, is connected to the patient port 12. The maximum inspiratory pressure (PIP), volume of the expired breath (VTe), respiratory rate (RR) and carbon dioxide concentration at the end of the expiration (etCO₂) are displayed on the display unity 7 as large numbers, which can be easily read under all environmental conditions. The display unit 7 is designed as a touch screen, so that the mode can be changed, e.g., from resuscitation mode to the respiration mode directly on the display surface. The electric connection is brought about via cable 6. In addition, sensor unit 8 has two buttons 16 for triggering mechanical breaths during the pause of the heart compressions during resuscitation.

FIG. 5 illustrates the measuring systems of the sensor unit 8. An infrared optical measuring device 14 for the carbon dioxide concentration, a hot wire anemometer 15 for measuring the gas flow 15, and a pressure sensor, not shown more specifically, for measuring the airway pressure are provided.

While specific embodiments of the invention have been shown and described in detail to illustrate the application of the principles of the invention, it will be understood that the invention may be embodied otherwise without departing from such principles.

### APPENDIX

### List of Reference Numbers

1 Patient
2 User
3 Respirator
4 Oxygen cylinder
5 Breathing gas tube
6 Cable
7 Display unit
8 Sensor unit
9 Holding element
10 Manual breathing bag
11 Supply tube
12 Patient port
13 Manual breathing bag port
14 Infrared optical measuring device
15 Hot wire anemometer
16 Button
100 Respiration device

What is claimed is:

1. A respiration device comprising:
a respirator;
a sensor unit to be positioned close to a patient, said sensor unit comprising a display unit for measured parameters;
a breathing gas tube between said respirator and said sensor unit;
a bidirectional data connection between said sensor unit and said respirator; and
a configuration means for providing presettable display fields for displaying measured parameters on said display unit.

2. A respiration device in accordance with claim 1, wherein said configuration means is part of said sensor unit.

3. A respiration device in accordance with claim 1, wherein said configuration means is part of said respirator.

4. A respiration device in accordance with claim 1, wherein said display presents display fields on a display on the basis of one of Liquid Crystal (LC) and Light Emitting Diode (LED) technology.

5. A respiration device in accordance with claim 1, wherein said display fields comprise a first group of display fields provided for manual respiration and a second group of display fields provided for mechanical respiration.

6. A respiration device in accordance with claim 5, wherein a group of display fields is selected corresponding to a mode of respiration set on said respirator.

7. A respiration device in accordance with claim 1, wherein a sensor unit holder is provided on said respirator for receiving said sensor unit.

8. A respiration device in accordance with claim 7, further comprising a switching-on means for activating said respirator or said sensor unit when removing said sensor unit from said sensor unit holder.

9. A respiration device in accordance with claim 1, wherein a triggering means for triggering mechanical inspiration is provided on said sensor unit.

10. A respiration device comprising:
a respirator;
breathing gas tube connected to said respirator;
a sensor unit connected to said breathing gas tube adjacent to a patient connection breathing gas tube end, said sensor unit comprising a sensor for sensing a measurable parameter of breathing gas in said breathing gas tube, said sensor unit further comprising a display unit for providing a display of measured parameters;
a bidirectional data connection between said sensor unit and said respirator; and
a configuration device providing a plurality of display fields with one or more of said plurality of display fields being used for displaying one or more measured parameters on said display unit.

11. A respiration device in accordance with claim 10, wherein said configuration device comprises a program module in a microprocessor of said sensor unit or of said respirator, which analyzes a current form of respiration and selects one or a plurality of corresponding display fields or offers a selection of display fields, that are preset as suitable, for selection.

12. A respiration device in accordance with claim 11, wherein said display unit presents display fields on the display on the basis of one of Liquid Crystal (LC) and Light Emitting Diode (LED) technology.

13. A respiration device in accordance with claim 11, wherein said display fields comprise a first group of display fields provided for manual respiration and a second group of display fields provided for mechanical respiration.

14. A respiration device in accordance with claim 13, wherein one of said first group of display fields and said second group of display fields is selected corresponding to a mode of respiration set on said respirator.

15. A respiration device in accordance with claim 13, wherein a sensor unit holder is provided on said respirator for receiving said sensor unit to hold said sensor unit relative to said respirator.

16. A respiration device in accordance with claim 15, further comprising a switching-on means for activating said respirator or said sensor unit when removing said sensor unit from said sensor unit holder.

17. A respiration device in accordance with claim 13, wherein a triggering means for triggering mechanical inspiration is provided on said sensor unit.

18. A respiration device comprising:
a respirator with a mechanical system for feeding breathing gas to a patient;
breathing gas tube connected to said respirator and with an opposite patient connection breathing gas tube end;
a sensor unit connected to said breathing gas tube adjacent to said patient connection breathing gas tube end, said sensor unit comprising a sensor for sensing at least one of airway pressure, breathing gas flow and carbon dioxide concentration, said sensor unit further comprising a display unit for providing a display of measured parameters;
a bidirectional data connection between said sensor unit and said respirator; and
a configuration device comprising a program module in a microprocessor of said sensor unit or of said respirator and with an associated memory with a plurality of display fields with one or more of said plurality of display fields being used for displaying one or more measured parameters on said display unit, said configuration device selecting one or a plurality of display fields for displaying one or more measured parameters on said display unit or offering a selection of display fields for displaying one or more measured parameters on said display unit based on the selection of display fields.

19. A respiration device in accordance with claim 18, wherein said plurality of display fields comprise at least one of a maximum inspiratory pressure (PIP), a volume of expired breath (VTe), a respiratory rate (RR) and a carbon dioxide concentration at an end of expiration (etCO2).

20. A respiration device in accordance with claim 18, further comprising: a manual breathing bag connected to the breathing gas tube for manual respiration of the patient wherein the manual respiration presents different requirements from mechanical respiration as to measured variables and said display fields comprise a first group of display fields provided for manual respiration and a second group of display fields provided for mechanical respiration and a group of display fields is selected corresponding to a mode of respiration set.
